SEQUENCE LISTING (7 sequences)

SEQ ID NO: 1 - DNA, 24 bp, Artificial Sequence
  Synthesized
tcgtcgttttgtcgttttgtcgtt

SEQ ID NO: 2 - DNA, 24 bp, Artificial Sequence
  Synthesized
tgctgcttttgtgcttttgtgctt

SEQ ID NO: 3 - DNA, 20 bp, Artificial Sequence
  Synthesized
gggggacgatcgtcgggggg

SEQ ID NO: 4 - DNA, 24 bp, Artificial Sequence
  Synthesized
tcgtcgttttgtcgttttgtcgtt

SEQ ID NO: 5 - DNA, 51 bp, Artificial Sequence
  Synthesized
acaaacataacagtgttggcccttacccataatcaactcacaaacataaca

SEQ ID NO: 6 - DNA, 18 bp, Artificial Sequence
  Synthesized
tcgtcgttttgtcgtttt

SEQ ID NO: 7 - DNA, 18 bp, Artificial Sequence
  Synthesized
tcgtgcttttgtgctttt

(19) United States
(12) Patent Application Publication
(10) Pub. No.: US 2010/0040576 A1
(43) Pub. Date: Feb. 18, 2010

(54) Modified Oligonucleotides For The Treatment Of Hepatitis C Infection

(75) Inventors: Chia-Cheng Kao, Bloomington, IN (US); Kumar Ranjith Tharachaparamba, Bloomington, IN (US)

Correspondence Address: Peter G. Carroll;MEDLEN & CARROLL, LLP, 101 Howard Street,, Suite 350, San Francisco, CA, 94105, US

(73) Assignee: The Texas A&M University System

(21) Appl. No.: 12/336,223

(22) Filed: Dec. 16, 2008

Related U.S. Application Data
(60) Provisional application No. 61/008,046, filed on Dec. 18, 2007.

(51) Int. Cl. A61K 38/21; A61K 39/395; A61K 39/29

(52) U.S. Cl. 424/85.5; 424/85.4; 424/85.6; 424/85.7; 424/178.1; 424/221.1; 424/226.1; 424/227.1; 424/228.1

(57) ABSTRACT

The invention relates to compositions and methods related to the treatment of viral infection. In some embodiments, the invention relates to the treatment of hepatitis C viral infection. In further embodiments, the invention relates to methods of administering oligonucleotide compositions for treating viral infections. In still further embodiments, the invention relates to the administration of antiviral agents, corticosteroids and immunomodulatory agents. In additional embodiments, the invention relates to the manipulation of immunostimulatory motifs within the oligonucleotides.

### FIELD OF INVENTION

The invention relates to compositions and methods related to the treatment of viral infection. In some embodiments, the invention relates to the treatment of hepatitis C viral infection. In further embodiments, the invention relates to methods of administering oligonucleotide compositions for treating viral infections. In still further embodiments, the invention relates to the administration of antiviral agents, corticosteroids and immunomodulatory agents. In additional embodiments, the invention relates to the manipulation of immunostimulatory motifs within the oligonucleotides.

### BACKGROUND OF THE INVENTION

Hepatitis C virus (HCV), a member of the Flavivirideae family, contains a positive single-stranded RNA genome that also functions as mRNA. Approximately 4 million persons in the United States and probably more than 100 million people worldwide are infected with hepatitis C virus (HCV). The virus has the unique ability to cause persistent infection in susceptible hosts after parenteral or percutaneous transmission. The immunological correlates of protection and viral clearance as well as the pathogenesis of liver injury are yet to be defined. Nearly 70% to 80% of infected persons become chronic carriers, and chronic and progressive HCV infection carries significant morbidity and mortality (e.g., major cause of cirrhosis, end-stage liver disease, and liver cancer).

Currently, the only approved therapy for treatment of chronic HCV infection is a 24-48 week course of the combination of type I interferon (IFNα/β) and ribavirin with sustained viral clearance observed in 42% to 82% of treated individuals. In addition to being ineffective in certain patient populations, this treatment is problematic due to considerable deleterious side effects. Ribavirin causes dose-related hemolysis and anemia, with severe side effects frequently observed with combination therapy. In many cases, treatment has to be reduced to mono-therapy with interferon alone. The mechanism(s) of actions of IFN (or resistance to IFN) during antiviral therapy for HCV and other RNA viruses are not well understood, although such an understanding is desirable to guide development of new antiviral treatments. Thus, there is a need to identify compositions and methods for reducing or inhibiting HCV infection that have limited adverse affects and that are effective in a wider range of patient populations.

### SUMMARY OF THE INVENTION

The invention relates to compositions and methods related to the treatment of viral infection. In some embodiments, the invention relates to the treatment of hepatitis C viral infection. In further embodiments, the invention relates to methods of administering oligonucleotide compositions for treating viral infections. In still further embodiments, the invention relates to the administration of antiviral agents, corticosteroids and immunomodulatory agents. In additional embodiments, the invention relates to the manipulation of immunostimulatory motifs within the oligonucleotides.

In some embodiments, the invention relates to a method comprising: providing; a subject with symptoms of a viral infection, and a pharmaceutical composition comprising at least one single-stranded DNA oligonucleotide containing at least one immunostimulatory motif, and administering said pharmaceutical composition to said subject under conditions such that an immune response is generated. In further embodiments, the virus causing said viral infection is an RNA virus. In still further embodiments, the RNA virus is a virus that causes hepatitis C. In additional embodiments, said virus that causes hepatitis C is resistant to treatment with antivirals selected from the group consisting of modified interferon, ribavirin and nucleoside analogs. In some embodiments, the RNA virus is selected from the group consisting of hepatitis A virus, hepatitis B virus, hepatitis E virus, rhinovirus, enterovirus and coronavirus. In further embodiments, said single-stranded DNA oligonucleotide is modified to contain at least one phosphorothioate bond between the bases. In still further embodiments, said single-stranded DNA oligonucleotide is modified to contain at least one chemical modifier selected from the group consisting of hydrogen, alkyl, amino, thiol, carboxyl, amido, phosphate, polyphosphate, phosphothioate, halide, carbamate, glycol based substituents such as polyethylene glycol (PEG), purines and derivatized purines, pyrimidines and derivatized pyrimidines, fatty acids, amino acids and heterocyclic compounds such as oxygen- and nitrogen-containing heterocycles. In additional embodiments, said immunostimulatory motif is a CpG. In some embodiments, said immunostimulatory motif is not CpG. In further embodiments, said single-stranded DNA oligonucleotide is modified to enhance transport across a cell membrane. In still further embodiments, said single-stranded DNA oligonucleotide consists of the nucleotide sequence set forth as SEQ ID NO: 1. In still further embodiments, said single-stranded DNA oligonucleotide consists of the nucleotide sequence set forth as SEQ ID NO: 2. In additional embodiments, the invention further comprises administering a second pharmaceutical composition to said subject. In some embodiments, said second pharmaceutical composition is selected from the group consisting of antiviral agents, corticosteroids and immunomodulatory agents. In further embodiments, said antiviral agent is selected from the group consisting of bacavir, acyclovir, agenerase, amatadine, amprenavir, crixivan, delavirdine, denavir, didanosine, efavirenz, epivir, famciclovir, famvir, fortovase, hivid, indinavir, ribavirin, invirase, lamivudine, nelfinavir, nevirapine, norvir, oseltamivir, penciclovir, relenza, rescriptor, retrovir, ribavirin, ritonavir, saquinavir, stavudine, sustiva, symdine, symmetrel, tamiflu, valacyclovir, valtrex, videx, viracept, viramidine, viramune, zalcitabine, zerit, ziagen, zidovudine, zovirax, and zanamivir. In still further embodiments, said corticosteroid is selected from the group consisting of dexamethasone (Decadron), hydrocortisone, methylprednisolone (Medrol), prednisone, cortisone, betamethasone, and prednisolone. In additional embodiments, said immunomodulatory agent is selected from the group consisting of Interferon, Interferon-alpha, Interferon-beta, Interferon-gamma, Interferon gamma-1b, Pegylated Interferon-alpha, Pegylated Interferon-alpha-2a, Interferon Nonresponders, Pegylated Interferon Nonresponders, Pegylated Interferon-alpha-2b, Actimmune, Tysabri, Natalizumab, Xolair, Omalizumab, Neulasta, Pegfilgrastim, Neupogen, Filgrastim, Anakinra, Humira, Adalimumab, Enbrel, TNF, Etanercept, Alefacept, Remicade, Infliximab, Raptiva, Efalizumab, Thymoglobulin, Infergen, Muromaonab, Zenapax, Daclizumab, and Basiliximab. In some embodiments, administering is selected from the group consisting of subcutaneous, oral, intravenous, transdermal, and intranasal routes. In further embodiments, said single-stranded DNA oligonucleotide is conjugated with an emulsifier to extend the in vivo half-life of said oligonucleotide. In still further embodiments, the invention further comprises, prior to step ii), coupling of said single-stranded DNA oligonucleotide to a cell permeable peptide. In additional embodiments, said cell permeable peptide is selected from the group consisting of Tat, Penetratin, Buforin II, Transportan, MAP, K-FGF, Ku70, Prion, pVEC, Pep-1, SynB1, Pep-7 and HN-1. In some embodiments, the invention further comprises, prior to step ii), coupling of said single-stranded DNA oligonucleotide to an antibody or antibody fragment. In further embodiments, said antibody or antibody fragment is generated by immunization. In still further embodiments, at least a portion of said antibody is generated synthetically.

In some embodiments, the invention relates to a method comprising: providing; a subject at risk for a viral infection, and a pharmaceutical composition comprising a single-stranded DNA oligonucleotide selected from the group consisting of the nucleotide sequence set forth as SEQ ID NO: 1 and the nucleotide sequence set forth as SEQ ID NO: 2, and administering said pharmaceutical composition to said subject under conditions such that said infection is prevented. In further embodiments, the virus causing said viral infection is an RNA virus. In still further embodiments, the RNA virus is a virus that causes hepatitis C. In additional embodiments, said virus that causes hepatitis C is resistant to treatment with antivirals selected from the group consisting of modified interferon, ribavirin and nucleoside analogs. In some embodiments, the RNA virus is selected from the group consisting of hepatitis A virus, hepatitis B virus, hepatitis E virus, rhinovirus, enterovirus and coronavirus. In further embodiments, said single-stranded DNA oligonucleotides are modified to be nuclease resistant. In still further embodiments, said single-stranded DNA oligonucleotides are modified to contain at least one phosphorothioate bond between the bases. In additional embodiments, said single-stranded DNA oligonucleotide is modified to enhance transport across a cell membrane. In some embodiments, the invention further comprises administering a second pharmaceutical composition to said subject. In further embodiments, said second pharmaceutical composition is selected from the group consisting of antiviral agents, corticosteroids and immunomodulatory agents. In still further embodiments, said antiviral agent is selected from the group consisting of bacavir, acyclovir, agenerase, amatadine, amprenavir, crixivan, delavirdine, denavir, didanosine, efavirenz, epivir, famciclovir, famvir, fortovase, hivid, indinavir, ribavirin, invirase, lamivudine, nelfinavir, nevirapine, norvir, oseltamivir, penciclovir, relenza, rescriptor, retrovir, ribavirin, ritonavir, saquinavir, stavudine, sustiva, symdine, symmetrel, tamiflu, valacyclovir, valtrex, videx, viracept, viramidine, viramune, zalcitabine, zerit, ziagen, zidovudine, zovirax, and zanamivir. In additional embodiments, said corticosteroid is selected from the group consisting of dexamethasone (Decadron), hydrocortisone, methylprednisolone (Medrol), prednisone, cortisone, betamethasone, and prednisolone. In some embodiments, said immunomodulatory agent is selected from the group consisting of Interferon, Interferon-alpha, Interferon-beta, Interferon-gamma, Interferon gamma-1b, Pegylated Interferon-alpha, Pegylated Interferon-alpha-2a, Pegylated Interferon-alpha-2b, Interferon Nonresponders, Pegylated Interferon Nonresponders, Actimmune, Tysabri, Natalizumab, Xolair, Omalizumab, Neulasta, Pegfilgrastim, Neupogen, Filgrastim, Anakinra, Humira, Adalimumab, Enbrel, TNF, Etanercept, Alefacept, Remicade, Infliximab, Raptiva, Efalizumab, Thymoglobulin, Infergen, Muromaonab, Zenapax, Daclizumab, and Basiliximab. In further embodiments, administering is selected from the group consisting of subcutaneous, oral, intravenous, transdermal, and intranasal routes. In still further embodiments, said single-stranded DNA oligonucleotides are conjugated with an emulsifier to extend said the in vivo half-life of said oligonucleotides. In additional embodiments, the invention further comprises, prior to step ii), coupling of said single-stranded DNA oligonucleotide to a cell permeable peptide. In some embodiments, said cell permeable peptide is selected from the group consisting of Tat, Penetratin, Buforin II, Transportan, MAP, K-FGF, Ku70, Prion, pVEC, Pep-1, SynB1, Pep-7 and HN-1. In further embodiments, the invention further comprises, prior to step ii), coupling of said single-stranded DNA oligonucleotide to an antibody or antibody fragment. In still further embodiments, said antibody or antibody fragment is generated by immunization. In additional embodiments, at least a portion of said antibody is generated synthetically.

In some embodiments, the invention relates to a method comprising: providing a virally infected subject that is asymptomatic, and a pharmaceutical composition comprising at least one single-stranded DNA oligonucleotide containing at least one immunostimulatory motif, and administering said pharmaceutical composition to said subject under conditions such that an immune response is generated. In further embodiments, the virus causing said viral infection is an RNA virus. In still further embodiments, the RNA virus is a virus that causes hepatitis C. In additional embodiments, said virus that causes hepatitis C is resistant to treatment with antivirals selected from the group consisting of modified interferon, ribavirin and nucleoside analogs. In some embodiments, the RNA virus is selected from the group consisting of hepatitis A virus, hepatitis B virus, hepatitis E virus, rhinovirus, enterovirus and coronavirus. In further embodiments, said single-stranded DNA oligonucleotides are modified to be nuclease resistant. In still further embodiments, said single-stranded DNA oligonucleotides are modified to contain at least one phosphorothioate bond between the bases. In additional embodiments, said single-stranded DNA oligonucleotide is modified to enhance transport across a cell membrane. In some embodiments, the invention further comprises administering a second pharmaceutical composition to said subject. In further embodiments, said second pharmaceutical composition is selected from the group consisting of antiviral agents, corticosteroids and immunomodulatory agents. In still further embodiments, said antiviral agent is selected from the group consisting of bacavir, acyclovir, agenerase, amatadine, amprenavir, crixivan, delavirdine, denavir, didanosine, efavirenz, epivir, famciclovir, famvir, fortovase, hivid, indinavir, ribavirin, invirase, lamivudine, nelfinavir, nevirapine, norvir, oseltamivir, penciclovir, relenza, rescriptor, retrovir, ribavirin, ritonavir, saquinavir, stavudine, sustiva, symdine, symmetrel, tamiflu, valacyclovir, valtrex, videx, viracept, viramidine, viramune, zalcitabine, zerit, ziagen, zidovudine, zovirax, and zanamivir. In additional embodiments, said corticosteroid is selected from the group consisting of dexamethasone (Decadron), hydrocortisone, methylpredniso lone (Medrol), prednisone, cortisone, betamethasone, and predniso lone. In some embodiments, said immunomodulatory agent is selected from the group consisting of Interferon, Interferon-alpha, Interferon-beta, Interferon-gamma, Interferon gamma-1b, Pegylated Interferon-alpha, Pegylated Interferon-alpha-2a, Pegylated Interferon-alpha-2b, Interferon Nonresponders, Pegylated Interferon Nonresponders, Actimmune, Tysabri, Natalizumab, Xolair, Omalizumab, Neulasta, Pegfilgrastim, Neupogen, Filgrastim, Anakinra, Humira, Adalimumab, Enbrel, TNF, Etanercept, Alefacept, Remicade, Infliximab, Raptiva, Efalizumab, Thymoglobulin, Infergen, Muromaonab, Zenapax, Daclizumab, and Basiliximab. In further embodiments, administering is selected from the group consisting of subcutaneous, oral, intravenous, transdermal, and intranasal routes. In still further embodiments, said single-stranded DNA oligonucleotides are conjugated with an emulsifier to extend said the in vivo half-life of said oligonucleotides. In additional embodiments, further comprises, prior to step ii), coupling of said single-stranded DNA oligonucleotide to a cell permeable peptide. In some embodiments, said cell permeable peptide is selected from the group consisting of Tat, Penetratin, Buforin II, Transportan, MAP, K-FGF, Ku70, Prion, pVEC, Pep-1, SynB1, Pep-7 and HN-1. In further embodiments, the invention further comprises, prior to step ii), coupling of said single-stranded DNA oligonucleotide to an antibody or antibody fragment. In still further embodiments, said antibody or antibody fragment is generated by immunization. In additional embodiments, at least a portion of said antibody is generated synthetically.

### DEFINITIONS

As used herein, a “virus” refers to a sub-microscopic infectious agent that is unable to grow or reproduce outside a host cell. Each viral particle, or virion, consists of genetic material, DNA or RNA, within a protective protein coat called a capsid. Their shape varies from simple helical and icosahedral (polyhedral or near-spherical) forms, to more complex structures with tails or an envelope. Viruses infect cellular forms of life and are grouped into animal, plant and bacterial viruses. Animal DNA viruses, such as rhinovirus, enterovirus, coronavirus, herpes and hepatitis viruses, often enter the host via endocytosis, the process by which cells take in material from the external environment. It is not intended that the invention be limited in any way by the method of infection or activity incorporated by the virus with respect to the infected host.

As used herein an “infection” refers to the presence of a virus in the body. Depending on the virus and the person's state of health, various viruses can infect almost any type of body tissue, from the brain to the skin. Viral infections cannot be treated with antibiotics; in fact, in some cases the use of antibiotics makes the infection worse. While many human viral infections can be effectively fought by the body's own immune system, the rest must be treated with anti-viral agents or other drugs.

As used herein a “nucleotide” refers to a chemical compound comprised of a heterocyclic base, a sugar and one or more phosphate groups. The base is a derivative of purine or pyrimidine and the sugar is a pentose, either deoxyribose or ribose. Nucleotides are the monomers of nucleic acids, with three or more bonding together to form a nucleic acid. “Oligonucleotides” are short sequences of nucleotides, either DNA or RNA, and typically comprise twenty or fewer nucleotidic bases under physiological settings. However, the synthesis of oligonucleotides using automated synthesizers allows for oligonucleotides of 160 to 200 bases or more.

As used herein a “pharmaceutical composition” is a pharmaceutically active compound that has been admixed with conventional pharmaceutical carriers and excipients (i.e., vehicles) and used in the form of aqueous solutions, tablets, capsules, elixirs, suspensions, syrups, wafers, and the like. Optionally, the pharmaceutical composition may contain other pharmaceutically acceptable components, such a buffers, surfactants, antioxidants, viscosity modifying agents, preservatives and the like. A liquid composition will generally consist of a suspension or solution of the compound or pharmaceutically acceptable salt in a suitable liquid carrier(s), for example, ethanol, glycerine, sorbitol, non-aqueous solvent such as polyethylene glycol, oils or water, with a suspending agent, preservative, surfactant, wetting agent, flavoring or coloring agent. Furthermore, a co-solvent, for example, polyethylene glycol, may be included in the formulation. All of these components are well known in the art.

As used herein, an “antibody” or “antibody fragment” is a protein or protein fragment that is used by the immune system to identify and neutralize foreign objects, such as bacteria and viruses. It is not intended that the present invention be limited to the means by which an antibody or antibody fragment is generated, for example, an antibody or antibody fragment may be generated through natural or non-natural means. They are made of a few basic structural units called chains; each antibody has two large heavy chains and two small light chains. Some antibodies are produced by a kind of white blood cell called a B cell. There are several different types of antibody heavy chain, and several different kinds of antibodies, which are grouped into different isotypes based on which heavy chain they possess.

As used herein an “immunomodulatory agent” refers to a pharmaceutically active compound that enhances, suppresses or otherwise affects a subject's immune system. Examples of these compounds include Interferon, Interferon-alpha, Interferon-beta, Interferon-gamma, Interferon gamma-1b, Pegylated Interferon-alpha, Pegylated Interferon-alpha-2a, Pegylated Interferon-alpha-2b, Interferon Nonresponders, Pegylated Interferon Nonresponders, Actimmune, Tysabri, Natalizumab, Xolair, omalizumab, Neulasta, Pegfilgrastim, Neupogen, Filgrastim, Anakinra, Humira, Adalimumab, Enbrel, TNF, Etanercept, Alefacept, Remicade, infliximab, Raptiva, Efalizumab, Thymoglobulin, Infergen, Muromaonab, Zenapax, Daclizumab, and Basiliximab. However, it is not intended that the present invention be limited to the use of any particular immunomodulatory agent.

As used herein, an “emulsifier” or “emulgent” is a substance that stabilizes an emulsion. Fatty acids, polyethylene glycol esters, e.g. PEG-20 stearate, and fatty acid/glycerine esters, e.g. glycerine stearate are examples of emulsifier. It is not intended that the present invention be limited to the selection of any particular emulsifier.

A “modification” refers to the technique of chemically reacting a protein or nucleic acid, e.g. an oligonucleotide, with chemical reagents. While it is not intended that the present invention be limited to modifications based solely on any one particular chemical substituent, preferred chemical substituents include hydrogen, alkyl, amino, thiol, carboxyl, amido, phosphate, polyphosphate, phosphothioate, halide, carbamate, glycol based substituents such as polyethylene glycol (PEG), purines and derivatized purines, pyrimidines and derivatized pyrimidines, fatty acids, amino acids and heterocyclic compounds such as oxygen- and nitrogen-containing heterocycles.

“Immunostimulatory motif” refers to a nucleotide pattern that induces activation or increases the activity of any of the components of the immune system. Examples of immunostimulatory motifs include but are not limited to AACGCC, AACGCT, AACGTC, AACGTT, AGCGCC, AGCGCT, AGCGTC, AGCGTT, GACGCC, GACGCT, GACGTC, GACGTT, GGCGCC, GGCGCT, GGCGTC, GGCGTT, ATCGCC, ATCGCT, ATCGTC, ATCGTT, GTCGCC, GTCGCT, GTCGTC, GTCGTT, TCGTCG, TCGTCGTCG, AACGCCCG, AACGCTCG, AACGTCCG, AACGTTCG, AGCGCCCG, AGCGCTCG, AGCGTCCG, AGCGTTCG, GACGCCCG, GACGCTCG, GACGTCCG, GACGTTCG, GGCGCCCG, GGCGCTCG, GGCGTCCG, GGCGTTCG, ATCGCCCG, ATCGCTCG, ATCGTCCG, ATCGTTCG, GTCGCCCG, GTCGCTCG, GTCGTCCG, and GTCGTTCG.

A “cell permeable peptide” refers to a peptide with the capacity to be transported to a cell's cytoplasm and/or nuclear compartments upon their introduction to an organism. Examples of cell permeable peptides include but are not limited to Tat, Penetratin, Buforin II, Transportan, MAP, K-FGF, Ku70, Prion, pVEC, Pep-1, SynB1, Pep-7 and HN-1.

### BRIEF DESCRIPTION OF THE DRAWINGS

FIG. 1 shows fluorescent micrographs of the interaction between the oligonucleotides ODN2006 and ODN2116 on hepatitis C virus (HCV) infection in FT3-7 cells. The green entity represents the HCV core protein.

FIG. 2 shows fluorescent micrographs of the interaction between the oligonucleotides ODN2006 and ODN2116 on hepatitis C virus (HCV) infection in Huh7.5 cells. The green entity represents the HCV core protein.

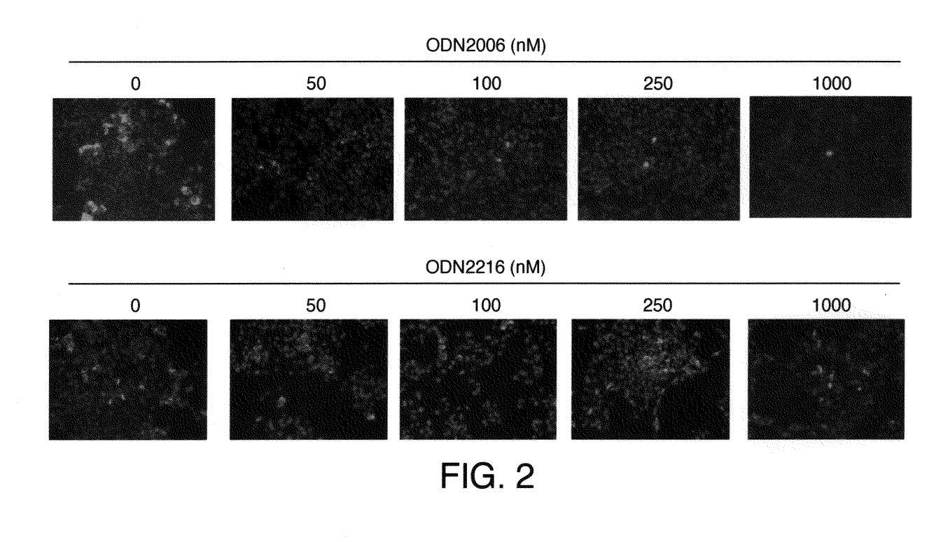

FIG. 3 shows a graph of the effects of the oligonucleotides ODN2006, ODN2116 and ODN2116 on HCV titers for Huh 7.5 cells in vitro.

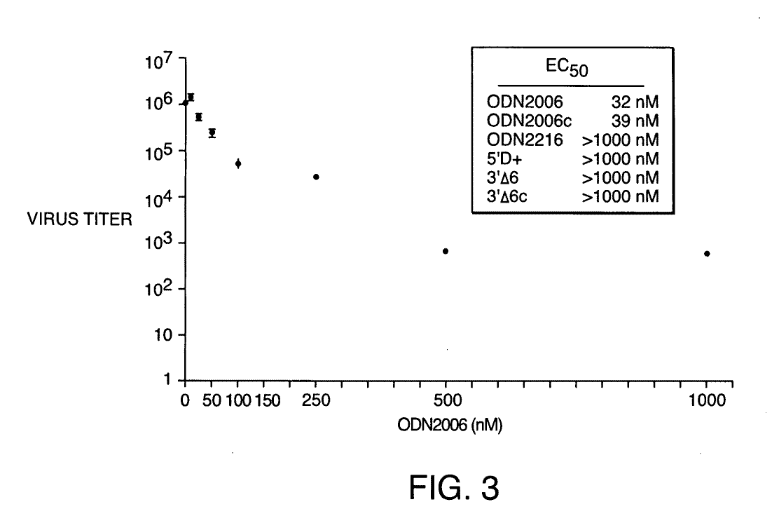

FIG. 4 shows Western blot analyses demonstrating the effects of ODNs on HCV expression.

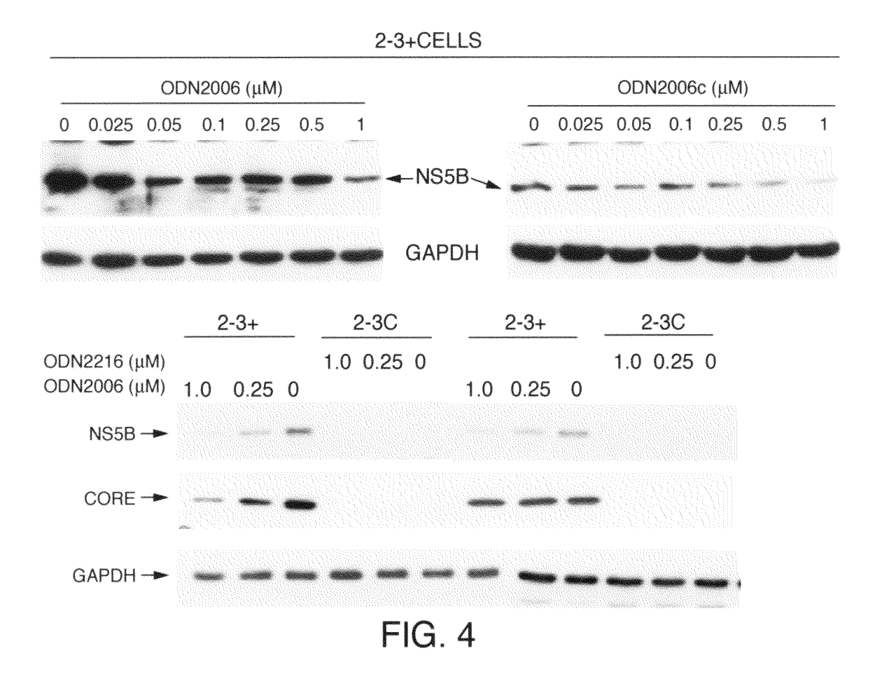

### DETAILED DESCRIPTION OF THE INVENTION

The invention relates to compositions and methods related to the treatment of viral infection. In some embodiments, the invention relates to the treatment of hepatitis C viral infection. In further embodiments, the invention relates to methods of administering oligonucleotide compositions for treating viral infections. In still further embodiments, the invention relates to the administration of antiviral agents, corticosteroids and immunomodulatory agents. In additional embodiments, the invention relates to the manipulation of immunostimulatory motifs within the oligonucleotides.

In some embodiments, the invention relates to the use of a pharmaceutical composition comprising at least one single-stranded DNA oligonucleotide containing at least one immunostimulatory motif. Modified single-stranded oligonucleotides can inhibit the infection of two HCV isolates in cultured cells. The inhibition is dependent on the concentration and the modification state of the oligonucleotide. While not intending to limit the present invention to any particular mechanism, it is believed that the oligonucleotides do not require a CpG motif for potent inhibitory activity, indicating that they do not act through signaling by Toll-like receptor 9. The inhibitors also inhibited the infection of two different strains of HCV. The effect of ODNs on cytokine production indicates the potential for a significant effect on viral infections, such as hepatitis C virus (HCV).

In some embodiments, the invention relates to the use of single-stranded DNA oligonucleotides that are modified to be nuclease resistant. As described in Bjersing, J. L. et al., Inflammation 28, 39-51 (2004), immunostimulatory unmethylated CpG-containing DNA with nuclease-protected phosphorothioate oligodeoxynucleotides are well known in the art. Advantages in using such motifs include the induction of the immune response of the host, thus the use of immunomodulatory nucleic acid molecules will not substantially result in the selection of resistant organisms. Additionally, immunomodulatory nucleic acids acts have been found to act in synergy with pharmaceuticals such as antivirals and conventional antibiotics as disclosed in U.S. Pat. No. 6,552,006 to Raz et al.

Innate immune receptors are promising targets to regulate the complex cascade of events that will lead to cytokine production. These receptors recognize pathogenic and endogenous ligands through their molecular signatures and then use several signaling pathways to alter gene expression. The subsequent production of cytokines and chemokines can dramatically affect the outcome of viral infections.

The Toll-like Receptors (TLRs) are a family of structurally related class I single pass transmembrane proteins that serve as the sentries for pathogen infections. At least eleven TLRs have been identified in the mammalian genome and are classified by the ligands that initially activate TLR-dependent signaling, including highly conserved pathogen proteins, cell wall components, and nucleic acids.

There are four TLRs that respond to nucleic acids. TLR7 and TLR8 recognize single-stranded (ss) RNAs, TLR9 recognizes ssDNA molecules that contain hypomethylated CpG motifs and TLR3 recognizes double-stranded (ds) RNAs. Modulating the signaling initiated by these TLRs have been demonstrated to affect the responses of TLRs. Examples of this modulation include: 1) CpG-containing DNAs could activate STAT1 and Interferon β in mouse macrophage and dendritic cells; 2) oligonucleotides consisting of homothymidines inhibited TLR7-specific signaling that was induced with Resiquimod; 3) Oligonucleotides that lack CpG motifs could suppress IL-8 production in human keratinocytes. There is currently a high level of interest to regulate the inflammation response to ameliorate the effects of cytokines and change the outcome of inflammation and diseases such as colitis, Crohn's disease, asthma, and sepsis.

Several TLRs can also regulate each other's signaling either through heterodimerization or through crosstalk mediated by ligands and/or signaling molecules. Modified ssDNA oligonucleotides (ODNs) are capable of inhibiting TLR3 activation of signaling in human cell lines as well as human peripheral blood mononuclear cells. This effect is does not require that the ODNs act on TLR9 since ODNs that do not activate TLR9 nonetheless have potent inhibitory activity on the TLR3 pathway. Whether other innate immunity receptors are positively or negatively affected by the ODNs that negatively regulate TLR3 is currently unknown.

### EXPERIMENTAL

The following examples are provided in order to demonstrate and further illustrate certain preferred embodiments and aspects of the present invention and are not to be construed as limiting the scope thereof.

In the experimental disclosure which follows, the following abbreviations apply: N (normal); M (molar); mM (millimolar); μM (micromolar); mol (moles); mmol (millimoles); μmol (micromoles); nmol (nanomoles); μmol (picomoles); g (grams); mg (milligrams); μg (micrograms); ng (nanograms); 1 or L (liters); ml (milliliters); μl (microliters); cm (centimeters); mm (millimeters); μm (micrometers); nm (nanometers); C (degrees Centigrade); IFN (interferon); HCV (hepatitis C virus); miR or miRNA (microRNA); MOI (multiplicity of infection); PCR (polymerase chain reaction).

### Example 1

### Materials and Methods

### Oligonucleotides

Modified oligonucleotides (ODNs) used: ODN2006, ODN2006c, and ODN2116 were purchased from InvivoGen (www.invivogen.com). ODN2006 and ODN2216 are ligands for Toll-like Receptor 9 due to the presence of CpG motifs (underlined below). ODN2006c is not a ligand of TLR9. The sequences of the ODNs used in this study are listed below, wherein an “s” denotes the presence of a phosphothioate bond and the lack of an “s” denotes the presence of a normal phosphodiester group. The number of bases in each ODN is shown in parentheses.

ODN2006:
5′-TsCsGsTsCsGsTsTsTsTsGsTsCsGsTsTsTsTsGsTsCsGs
TsT-3′ (24-nt)
ODN2006c:
5′-TsGsCsTsGsCsTsTsTsTsGsTsGsCsTsTsTsTsGsTsGsCs
TsT-3′ (24-nt)
ODN2216:
5′-GsGsGGGACGATCGTCGsGsGsGsGsG-3′ (20-nt)
dODN2006: ODN2006:
5′-TCGTCGTTTTGTCGTTTTGTCGTT-3′ (24-nt)
5′D+:
AsCsAsAsAsCATAACAGTGTTGGCCCTTACCCATAATCAACTCACA
AA-CAsTsAsAsCsA (52-nt)
3′Δ6:
TsCsGsTCGTTTTGTCGTsTsTsT (18-nt)
3′Δ6c:
TsCsGsTGCTTTTGTGCTsTsTsT (18-nt)

### Viruses

The JFH1 virus is a genotype 2a HCV recovered from a Japanese patient with fulminant hepatitis C. It was the first HCV strain found to efficiently infect cells in culture and has served as a model for studies of HCV infection and for the identification of antivirals.

### Cells

Huh 7.5 cells are derived from human hepatocytes and can be infected by hepatitis C virus to yield higher virus titers due to a mutation in the intracellular helicase gene named RIG-I (22).

The FT3-7 cell line is derived from the Huh7 hepatocytes following transformation with a plasmid vector expressing Toll-like receptor 3 (TLR3). FT3-7 cells expresses a low abundance of TLR3 but, for reasons that remain unclear, have an increased capacity to produce infectious HCV in comparison to Huh-7.5 cells.

2-3+ cells are a Huh7-derived hepatoma cell line, which contains a full-length copy of replicating HCV N strain genome.

2-3c cells are derived from the 2-3+ cell line, wherein the HCV RNA has been eliminated by treatment with IFN-α2b.

### Examination of the Antiviral Effect of Modified Oligonucleotides (ODNS)

The effect of ODNs on HCV infection was determined using the Huh7.5 cell line. Cells were plated at a density of 2×10⁴ cells per well of an 8 well chamber. After 24 h ODNs were added to the cells at concentrations ranging from 10 nM to 5 μM, along with the virus HCV JFH1 at a MOI of 0.5. Each ODN concentration was performed in triplicate. Controls for this experiment included three wells of untreated cells, as well as three wells of cells treated with each ODN (described above). After 24 h, the medium was replaced with fresh medium containing the same concentrations of ODN. After 48 h the media supernatant was collected and stored. The cells were then harvested for immunofluorescent assay (IFA) using mouse monoclonal antibody to the HCV Core protein.

### Immunofluorescent Assay (IFA)

Cells were washed in 1×PBS and fixed with 4% PFA in PBS for 30′ at room temperature. Cells were then washed with PBS containing 100 mM glycine, permeabilized with 0.2% Triton-X100 in PBS for 15′, and blocked with 10% goat serum in PBS. Monoclonal antibody to the HCV Core protein was then added and incubated for 1 h. Unbound antibody was removed using an IFA wash solution (0.005% Tween-20 in 1×PBS) followed by incubation with a fluorescent-tagged antibody specific for the (mouse) Fc portion of the first antibody. Incubations were performed for 1 h, in the dark, at room temperature. Unbound antibody was removed with the IFA wash solution, followed by a 5′ incubation with DAPI (1:1000 in PBS), and a final PBS rinse. Media in the chambers was then removed, and one drop of Vectashield added to each chamber. A coverslip was then added the each well and sealed with nail polish. Fluorescent cells were then enumerated by microscopy.

### Determination of Half Maximum Effective Concentration (EC₅₀)

To determine the effective concentration of ODN needed to reduce infectious JFH-1 by 50%, the titer of virus released into the medium were titered after re-infecting Huh7.5 cells with dilutions of the medium. FT3-7 cells were used to determine the EC₅₀ for each ODN. 1.5×10⁴ cells were plated per well of an 8 well chamber. Approximately 100 virus particles along with 25 nM to 1 μM of ODNs were then added to the media in each well. IFA was performed as described above, and number of virus-infected cells was quantified. EC₅₀ values were determined based on the average number of infected cells at different ODN concentrations.

### Determination of HCV Titer

The titer of virus released into the media supernatants was determined based on the infecting inoculum and number of infected cells identified by the IFA assay described above.

### Western Blot Analysis

The effect of ODNs on viral protein expression was determined using the Huh7 hepatoma cell lines; NNeo/C-5B clones 2-3+, which contain replicating genome-length RNA expressing all of the proteins of the HCV-N strain, and 2-3c in which HCV RNA had been eliminated by prior treatment with IFN-α2b. Cell lines 2-3+ and 2-3c were transfected with ODNs using lipofectamine, and harvested 48 hours later by cells lysis. Western blots of the lysed cells were then probed sing antibodies to both the HCV NS5B protein and the HCV Core protein.

### Results

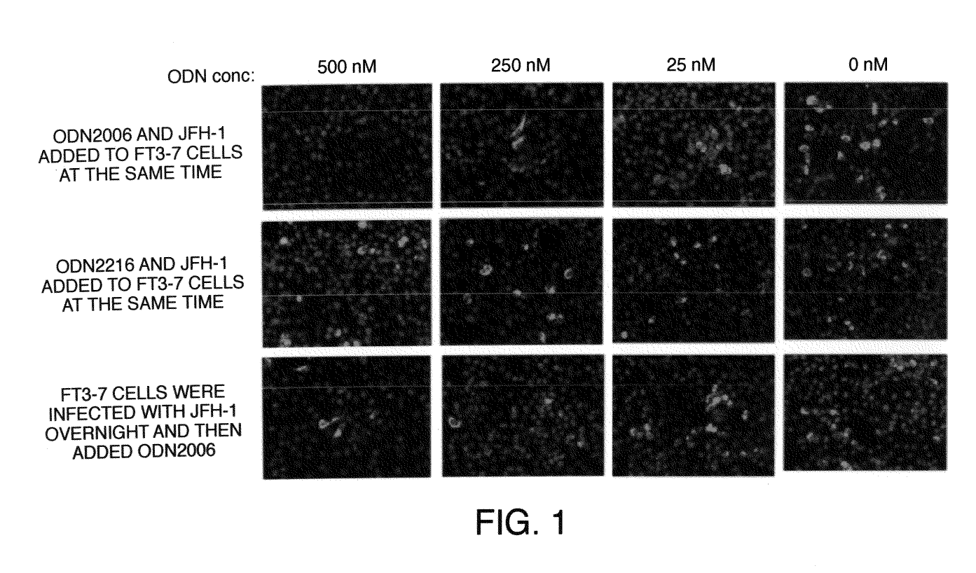

The presence of ODN2006 had a dramatic decrease in JFH-I infection (FIG. 1). In addition, the decrease in infection was dependent on ODN2006 concentration (FIG. 1). The effects of ODN2006 are also specific since another ODN, ODN2216, did not decrease JFH-1 infection in FT3-7 cells (FIG. 1). Given that both ODN2006 and ODN2216 are ligands for TLR9, these results suggest that the inhibitory effect is not exerted through TLR9. It was also observed that ODN2006 did not obviously affect adenovirus infection in 293T cells (derived from a human embryonic kidney cells) and that ODN2006 had a less than two-fold effect on the infection of a positive-stranded RNA Coronavirus, Mouse Hepatitis Virus (data not shown). These results indicate that ODN2006 contains features that could inhibit HCV infection in hepatocyte-derived cell line.

To examine whether the inhibition of JFH-1 infection was at the level of virus entry, FT3-7 cells were infected for 8 h prior to the addition of ODN2006. Core protein level was detected at 40 h later and a concentration-dependent inhibition of JFH-1 was again observed, although higher concentrations of ODN2006 were needed when the virus has an 8 h head start. Nonetheless, results indicate that ODN2006 is unlikely to be solely blocking HCV infection at the level of virus entry.

Experiments examining whether ODN2006 can affect HCV infection in Huh7.5 cells indicate that in the presence of even 50 nM of ODN2005, JFH-1 infection was reduced to less than half of the level seen in the absence of ODN2006. Consistent with the results in FT3-7 cells, ODN2216 did not have an inhibitory effect even at 1 μM, the highest concentration tested.

Experiments directed at determining the effective concentration of ODN needed to reduce infectious JFH-1 by 50% demonstrated the EC₅₀ for ODN2006 to be 32 nM; while the EC50 of ODN2006c, which lacks the CpG motifs found in ODN2006 and is not a TLR9 ligand, was determined to be 39 nM. This confirms that motifs within the ODN required to activate TLR9 signaling are not essential for the inhibition of HCV infection.

Additional ODNs, including ODN2116, 3′Δ6, 3′Δ6c, and dODN2006, which is identical to ODN2006 except that all of the phosphothioates were replaced with phosphodiesters, were found to have no observable effects on HCV infection of FT3-7 cells at a concentration of 1000 nM. Thus, a modified DNA backbone is required for the inhibitory activity of ODNs and a particular base sequence is not sufficient for inhibitory activity. Phosphorothioates are known to affect the degradation rate of nucleic acid mimetics in body fluids and it is possible that their effect may be related to the turnover of the ODNs.

Experiments to determine whether ODN2006 and ODN2006c could inhibit HCV demonstrated that both ODN2006 and ODN2006c reproducibly reduced the abundance of both NS5B and the Core protein while having no obvious effects on the level of GAPDH. Inhibition of Core protein production in FT2 was also confirmed by microscopy in cells stained with antibody detecting the Core protein.

It is anticipated that other man-made or natural modifications in the termini, the lengths, the bases, and the phosphodiesters of the ODNs can modulate the inhibitory activities of the ODNs. These modifications could improve the inhibitory activities of the ODNS through improved binding to the receptor, changes in the rate of normal metabolism of the ODNs, or changes in the localization in cells and tissues. Identification of the receptor required for select ODNs to inhibit HCV infection and the identification of the motifs required within ODNs will enhance this capability. The use of safe and effective antiviral inhibitors derived from ODNs can be practiced in parallel with studies to obtain a detailed understanding of the mechanism of action of the inhibitory ODNs. The ability of ODNs that interact with the innate immunity receptors to inhibit HCV infection in cultured cells provides proof of principal in the development of this class of HCV inhibitors.

We claim:

1. A method comprising:
i) providing;
a) a subject with symptoms of a viral infection, and
b) a pharmaceutical composition comprising at least one single-stranded DNA oligonucleotide containing at least one immunostimulatory motif, and;
ii) administering said pharmaceutical composition to said subject under conditions such that an immune response is generated.

2. The method of claim 1, wherein the virus causing said viral infection is an RNA virus.

3. The method of claim 2, wherein the RNA virus is a virus that causes hepatitis C.

4. The method of claim 3, wherein said virus that causes hepatitis C is resistant to treatment with antivirals selected from the group consisting of modified interferon, ribavirin and nucleoside analogs.

5. The method of claim 2, wherein the RNA virus is selected from the group consisting of hepatitis A virus, hepatitis B virus, hepatitis E virus, rhinovirus, enterovirus and coronavirus.

6. The method of claim 1, wherein said single-stranded DNA oligonucleotide is modified to contain at least one phosphorothioate bond between the bases.

7. The method of claim 1, wherein said single-stranded DNA oligonucleotide is modified to contain at least one chemical modifier selected from the group consisting of hydrogen, alkyl, amino, thiol, carboxyl, amido, phosphate, polyphosphate, phosphothioate, halide, carbamate, glycol based substituents such as polyethylene glycol (PEG), purines and derivatized purines, pyrimidines and derivatized pyrimidines, fatty acids, amino acids and heterocyclic compounds such as oxygen- and nitrogen-containing heterocycles.

8. The method of claim 1, wherein said immunostimulatory motif is a CpG.

9. The method of claim 1, wherein said immunostimulatory motif is not CpG.

10. The method of claim 1, wherein said single-stranded DNA oligonucleotide is modified to enhance transport across a cell membrane.

11. The method of claim 1, wherein said single-stranded DNA oligonucleotide consists of the nucleotide sequence set forth as SEQ ID NO: 1.

12. The method of claim 1, wherein said single-stranded DNA oligonucleotide consists of the nucleotide sequence set forth as SEQ ID NO: 2.

13. The method of claim 1, further comprising administering a second pharmaceutical composition to said subject.

14. The method of claim 13, wherein said second pharmaceutical composition is selected from the group consisting of antiviral agents, corticosteroids and immunomodulatory agents.

15. The method of claim 14, wherein said antiviral agent is selected from the group consisting of bacavir, acyclovir, agenerase, amatadine, amprenavir, crixivan, delavirdine, denavir, didanosine, efavirenz, epivir, famciclovir, famvir, fortovase, hivid, indinavir, ribavirin, invirase, lamivudine, nelfinavir, nevirapine, norvir, oseltamivir, penciclovir, relenza, rescriptor, retrovir, ribavirin, ritonavir, saquinavir, stavudine, sustiva, symdine, symmetrel, tamiflu, valacyclovir, valtrex, videx, viracept, viramidine, viramune, zalcitabine, zerit, ziagen, zidovudine, zovirax, and zanamivir.

16. The method of claim 14, wherein said corticosteroid is selected from the group consisting of dexamethasone (Decadron), hydrocortisone, methylprednisolone (Medrol), prednisone, cortisone, betamethasone, and prednisolone.

17. The method of claim 14, wherein said immunomodulatory agent is selected from the group consisting of Interferon, Interferon-alpha, Interferon-beta, Interferon-gamma, Interferon gamma-1b, Pegylated Interferon-alpha, Pegylated Interferon-alpha-2a, Interferon Nonresponders, Pegylated Interferon Nonresponders, Pegylated Interferon-alpha-2b, Actimmune, Tysabri, Natalizumab, Xolair, Omalizumab, Neulasta, Pegfilgrastim, Neupogen, Filgrastim, Anakinra, Humira, Adalimumab, Enbrel, TNF, Etanercept, Alefacept, Remicade, Infliximab, Raptiva, Efalizumab, Thymoglobulin, Infergen, Muromaonab, Zenapax, Daclizumab, and Basiliximab.

18. The method of claim 1, wherein administering is selected from the group consisting of subcutaneous, oral, intravenous, transdermal, and intranasal routes.

19. The method of claim 1, wherein said single-stranded DNA oligonucleotide is conjugated with an emulsifier to extend the in vivo half-life of said oligonucleotide.

20. The method of claim 1, further comprising, prior to step ii), coupling of said single-stranded DNA oligonucleotide to a cell permeable peptide.

21. The method of claim 20, wherein said cell permeable peptide is selected from the group consisting of Tat, Penetratin, Buforin II, Transportan, MAP, K-FGF, Ku70, Prion, pVEC, Pep-1, SynB1, Pep-7 and HN-1.

22. The method of claim 1, further comprising, prior to step ii), coupling of said single-stranded DNA oligonucleotide to an antibody or antibody fragment.

23. The method of claim 22, wherein said antibody or antibody fragment is generated by immunization.

24. The method of claim 22, wherein at least a portion of said antibody is generated synthetically.

25. A method comprising:
i) providing;
a) a subject at risk for a viral infection, and
b) a pharmaceutical composition comprising a single-stranded
DNA oligonucleotide selected from the group consisting of the nucleotide sequence set forth as SEQ ID NO: 1 and the nucleotide sequence set forth as SEQ ID NO: 2, and;
ii) administering said pharmaceutical composition to said subject under conditions such that said infection is prevented.

26. The method of claim 25, wherein the virus causing said viral infection is an RNA virus.

27. The method of claim 26, wherein the RNA virus is a virus that causes hepatitis C.

28. The method of claim 27, wherein said virus that causes hepatitis C is resistant to treatment with antivirals selected from the group consisting of modified interferon, ribavirin and nucleoside analogs.

29. The method of claim 26, wherein the RNA virus is selected from the group consisting of hepatitis A virus, hepatitis B virus, hepatitis E virus, rhinovirus, enterovirus and coronavirus.

30. The method of claim 25, wherein said single-stranded DNA oligonucleotides are modified to be nuclease resistant.

31. The method of claim 25, wherein said single-stranded DNA oligonucleotides are modified to contain at least one phosphorothioate bond between the bases.

32. The method of claim 25, wherein said single-stranded DNA oligonucleotide is modified to enhance transport across a cell membrane.

33. The method of claim 25, further comprising administering a second pharmaceutical composition to said subject.

34. The method of claim 33, wherein said second pharmaceutical composition is selected from the group consisting of antiviral agents, corticosteroids and immunomodulatory agents.

35. The method of claim 34, wherein said antiviral agent is selected from the group consisting of bacavir, acyclovir, agenerase, amatadine, amprenavir, crixivan, delavirdine, denavir, didanosine, efavirenz, epivir, famciclovir, famvir, fortovase, hivid, indinavir, ribavirin, invirase, lamivudine, nelfinavir, nevirapine, norvir, oseltamivir, penciclovir, relenza, rescriptor, retrovir, ribavirin, ritonavir, saquinavir, stavudine, sustiva, symdine, symmetrel, tamiflu, valacyclovir, valtrex, videx, viracept, viramidine, viramune, zalcitabine, zerit, ziagen, zidovudine, zovirax, and zanamivir.

36. The method of claim 34, wherein said corticosteroid is selected from the group consisting of dexamethasone (Decadron), hydrocortisone, methylprednisolone (Medrol), prednisone, cortisone, betamethasone, and prednisolone.

37. The method of claim 34, wherein said immunomodulatory agent is selected from the group consisting of Interferon, Interferon-alpha, Interferon-beta, Interferon-gamma, Interferon gamma-1b, Pegylated Interferon-alpha, Pegylated Interferon-alpha-2a, Pegylated Interferon-alpha-2b, Interferon Nonresponders, Pegylated Interferon Nonresponders, Actimmune, Tysabri, Natalizumab, Xolair, Omalizumab, Neulasta, Pegfilgrastim, Neupogen, Filgrastim, Anakinra, Humira, Adalimumab, Enbrel, TNF, Etanercept, Alefacept, Remicade, Infliximab, Raptiva, Efalizumab, Thymoglobulin, Infergen, Muromaonab, Zenapax, Daclizumab, and Basiliximab.

38. The method of claim 25, wherein administering is selected from the group consisting of subcutaneous, oral, intravenous, transdermal, and intranasal routes.

39. The method of claim 25, wherein said single-stranded DNA oligonucleotides are conjugated with an emulsifier to extend said the in vivo half-life of said oligonucleotides.

40. The method of claim 25, further comprising, prior to step ii), coupling of said single-stranded DNA oligonucleotide to a cell permeable peptide.

41. The method of claim 40, wherein said cell permeable peptide is selected from the group consisting of Tat, Penetratin, Buforin II, Transportan, MAP, K-FGF, Ku70, Prion, pVEC, Pep-1, SynB1, Pep-7 and HN-1.

42. The method of claim 25, further comprising, prior to step ii), coupling of said single-stranded DNA oligonucleotide to an antibody or antibody fragment.

43. The method of claim 42, wherein said antibody or antibody fragment is generated by immunization.

44. The method of claim 42, wherein at least a portion of said antibody is generated synthetically.

45. A method comprising:
i) providing;
a) a virally infected subject that is asymptomatic, and
b) a pharmaceutical composition comprising at least one single-stranded DNA oligonucleotide containing at least one immunostimulatory motif, and;
ii) administering said pharmaceutical composition to said subject under conditions such that an immune response is generated.

46. The method of claim 45, wherein the virus causing said viral infection is an RNA virus.

47. The method of claim 46, wherein the RNA virus is a virus that causes hepatitis C.

48. The method of claim 47, wherein said virus that causes hepatitis C is resistant to treatment with antivirals selected from the group consisting of modified interferon, ribavirin and nucleoside analogs.

49. The method of claim 46, wherein the RNA virus is selected from the group consisting of hepatitis A virus, hepatitis B virus, hepatitis E virus, rhinovirus, enterovirus and coronavirus.

50. The method of claim 45, wherein said single-stranded DNA oligonucleotides are modified to be nuclease resistant.

51. The method of claim 45, wherein said single-stranded DNA oligonucleotides are modified to contain at least one phosphorothioate bond between the bases.

52. The method of claim 45, wherein said single-stranded DNA oligonucleotide is modified to enhance transport across a cell membrane.

53. The method of claim 45, further comprising administering a second pharmaceutical composition to said subject.

54. The method of claim 53, wherein said second pharmaceutical composition is selected from the group consisting of antiviral agents, corticosteroids and immunomodulatory agents.

55. The method of claim 54, wherein said antiviral agent is selected from the group consisting of bacavir, acyclovir, agenerase, amatadine, amprenavir, crixivan, delavirdine, denavir, didanosine, efavirenz, epivir, famciclovir, famvir, fortovase, hivid, indinavir, ribavirin, invirase, lamivudine, nelfinavir, nevirapine, norvir, oseltamivir, penciclovir, relenza, rescriptor, retrovir, ribavirin, ritonavir, saquinavir, stavudine, sustiva, symdine, symmetrel, tamiflu, valacyclovir, valtrex, videx, viracept, viramidine, viramune, zalcitabine, zerit, ziagen, zidovudine, zovirax, and zanamivir.

56. The method of claim 54, wherein said corticosteroid is selected from the group consisting of dexamethasone (Decadron), hydrocortisone, methylprednisolone (Medrol), prednisone, cortisone, betamethasone, and prednisolone.

57. The method of claim 54, wherein said immunomodulatory agent is selected from the group consisting of Interferon, Interferon-alpha, Interferon-beta, Interferon-gamma, Interferon gamma-1b, Pegylated Interferon-alpha, Pegylated Interferon-alpha-2a, Pegylated Interferon-alpha-2b, Interferon Nonresponders, Pegylated Interferon Nonresponders, Actimmune, Tysabri, Natalizumab, Xolair, Omalizumab, Neulasta, Pegfilgrastim, Neupogen, Filgrastim, Anakinra, Humira, Adalimumab, Enbrel, TNF, Etanercept, Alefacept, Remicade, Infliximab, Raptiva, Efalizumab, Thymoglobulin, Infergen, Muromaonab, Zenapax, Daclizumab, and Basiliximab.

58. The method of claim 45, wherein administering is selected from the group consisting of subcutaneous, oral, intravenous, transdermal, and intranasal routes.

59. The method of claim 45, wherein said single-stranded DNA oligonucleotides are conjugated with an emulsifier to extend said the in vivo half-life of said oligonucleotides.

60. The method of claim 45, further comprising, prior to step ii), coupling of said single-stranded DNA oligonucleotide to a cell permeable peptide.

61. The method of claim 60, wherein said cell permeable peptide is selected from the group consisting of Tat, Penetratin, Buforin II, Transportan, MAP, K-FGF, Ku70, Prion, pVEC, Pep-1, SynB1, Pep-7 and HN-1.

62. The method of claim 45, further comprising, prior to step ii), coupling of said single-stranded DNA oligonucleotide to an antibody or antibody fragment.

63. The method of claim 62, wherein said antibody or antibody fragment is generated by immunization.

64. The method of claim 62, wherein at least a portion of said antibody is generated synthetically.
